(19) United States
(12) Patent Application Publication
(10) Pub. No.: US 2023/0248846 A1
(43) Pub. Date: Aug. 10, 2023

(54) MINI-PROMOTER

(71) Applicant: Vanderbilt University, Nashville, TN (US)

(72) Inventors: Tonia S. Rex, Nashville, TN (US); Jon R. Backstrom, Nashville, TN (US)

(21) Appl. No.: 18/065,360

(22) Filed: Dec. 13, 2022

Related U.S. Application Data
(60) Provisional application No. 63/288,977, filed on Dec. 13, 2021.

(51) Int. Cl. A61K 48/00; C12N 15/86

(52) U.S. Cl. CPC A61K 48/0058; C12N 15/86; C12N 2750/14143; C12N 2750/14145; C12N 2750/14171; C12N 2830/008

(57) ABSTRACT

A promoter for modulating expression of a target gene from a vector includes an enhancer portion comprising the nucleotide sequence of SEQ ID NO: 1 or its complement; a core promoter portion, containing an RNA polymerase binding site, a TATA box, and a transcription start site; and a linker disposed between the enhancer portion and the core promoter portion, containing a restriction site.

### RELATED APPLICATIONS

This application claims priority from U.S. Provisional Application Ser. No. 63/288,977 filed Dec. 13, 2021, the entire disclosure of which is incorporated herein by this reference.

### TECHNICAL FIELD

The presently-disclosed subject matter relates to modulation of gene expression in a targeted cell. In particular, the presently-disclosed subject matter relates to a mini-promoter for use in modulating expression of a target gene in certain glial cells, such as astrocytes and Muller cells.

### INTRODUCTION

Glial cells are found through the nervous system and include various types, such as astrocytes, oligodendrocytes, microglia, Muller glia, Schwann/satellite glia, and enteric glia. The various glial cell types are found in different areas of the central nervous system and peripheral nervous system, and have distinct roles. For example, astrocytes support the blood-brain barrier, provide nutrients to neurons, and play a crucial role in maintaining extracellular ion balance and neurotransmitter levels in the CNS (O'Carroll, 2021). For another example, the retina contains Müller cells which, similar to astrocytes, help to regulate blood flow, uptake of neurotransmitters, regulation of ion levels, and energy storage. Glial cells play roles in neurodegenerative diseases and have been proposed as gene therapy targets for treating these conditions. However, the ability to target a particular glial cell type remains elusive and presents an obstacle to providing effective gene therapy options. Accordingly, there remains a need in the art for tools that can specifically target a particular glial cell type, e.g., astrocytes, Muller cells, to modulate gene expression a target gene in the particular glial cell type.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes a promoter for modulating expression of a target gene from a vector, which comprises an enhancer portion comprising the nucleotide sequence of SEQ ID NO: 1 or its complement; a core promoter portion, containing an RNA polymerase binding site, a TATA box, and a transcription start site; and a linker disposed between the enhancer portion and the core promoter portion, containing a restriction site.

In some embodiments, the promoter increases expression of the target gene in astrocytes or Muller cells, while decreasing expression of the target gene in other cells. In some embodiments, the promoter increases expression of the target gene, relative to expression of the target gene when using a vimentin promoter without the enhancer portion.

In some embodiments of the promoter, the core promoter portion is derived from a core region of minimal vimentin promoter (minVim). In some embodiments, the core promoter portion comprises the nucleic acid sequence of SEQ ID NO: 2 or its complement. In some embodiments, the promoter increases expression of the target gene in cells that express vimentin and decreases expression of the target gene in cells that do not express vimentin.

In some embodiments of the promoter, the linker comprises the nucleic acid sequence of SEQ ID NO: 3. In some embodiments, the promoter comprises the nucleic acid sequence of SEQ ID NO: 4.

The presently-disclosed subject matter further includes a vector comprising a promoter as disclosed herein. In some embodiments, the vector is an adenovirus associated virus (AAV).

The presently-disclosed subject matter further includes a method of modulating expression of a target gene in a cell, which involves administering a vector for enhancing or suppressing expression of the target gene, which vector comprises a promoter as disclosed herein. In some embodiments, the vector is an AAV. In some embodiments, the target gene is involved in inflammatory or protective effects of reactive gliosis. In some embodiments, the cell is in a subject. In some embodiments, the subject has or has a risk of Alzheimer's disease or Parkinson's disease.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-IC. rAAV6quad.mVim specifically transduces astrocytes. Confocal micrographs of a retinal flat-mount after IVt injection of rAAVquadmut.mVim.mCherry showing complete overlap with GFAP positive astrocytes and no evidence of Müller cell endfeet.

FIGS. 2A and 2B. Analysis of endogenous vimentin expression. FIG. 2A includes a series of confocal micrographs. FIG. 2B includes Western blots, where 20 mg of cell extract was placed in each lane. Lanes: 1—mouse retinal primary astrocytes, 2—human retinal pigmented epithelia (ARPE-19), 3—human breast adenocarcinoma (MCF-7). The left panel is vimentin and the right panel is α-tubulin.

FIGS. 3A and 3B. Regions that regulate expression from the human vimentin promoter in different cell types. FIG. 3A includes depictions of promoter constructs (grey regions) that were transfected into ARPE-19, astrocytes, and MCF-7 cells. FIG. 3B compares the regulation of expression in astrocytes and MCF-7 cells.

FIG. 4. Vimentin promoter truncations with core promoter, including VZ-60 (SEQ ID NOS: 5 and 6), VZ-49 (SEQ ID NOS: 7 and 8), and VZ-50 (SEQ ID NOS: 9 and 10).

FIG. 5. Analysis of vimentin promoters in MCF-7 cells, including VZ-60 (SEQ ID NOS: 5 and 6), VZ-67 (SEQ ID NOS: 11 and 12), and VZ-68 (SEQ ID NOS: 13 and 14, and for VZ-60 (SEQ ID NOS: 5 and 6), VZ-70 (SEQ ID NOS: 15 and 16), and VZ-71 (SEQ ID NOS: 17 and 18).

FIG. 6. Depiction of wild type and mini-human vimentin promoters.

### REFERENCE TO AN ELECTRONIC SEQUENCE LISTING

The contents of the electronic sequence listing (Rex VU 21128 Sequence Listing st26.xml; Size: 17,687 bytes; and Date of Creation: Dec. 13, 2022) is herein incorporated by reference in its entirety.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter relates to modulation of gene expression, particularly in astrocytes and Muller cells. The presently-disclosed subject matter includes an mini-promoters for use in modulating expression of a target gene in astrocytes and Muller cells.

The presently-disclosed subject matter includes a promoter for modulating expression of a target gene from a vector, which includes an enhancer portion comprising the nucleotide sequence of SEQ ID NO: 1 or its complement; a core promoter portion, containing an RNA polymerase binding site, a TATA box, and a transcription start site; and a linker disposed between the enhancer portion and the core promoter portion, containing a restriction site. In some embodiments, the total length of the promoter is about 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 bp. In some embodiments, the promoter is 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, or 3.5 kb.

In some embodiments, the promoter having the enhancer portion increases expression of the target gene specifically in astrocytes, while decreasing expression of the target gene in other cells In some embodiments, the promoter with astrocyte specificity is about 2.5, 2.6, 2.7, 2.8, 2.9, 3, 3.1, 3.2, 3.3, 3.4, or 3.5 kb. In some embodiments, the promoter having the enhancer portion increases expression of the target gene in astrocytes or Muller cells. In some embodiments, the promoter is 1, 1.1, 1.2, 1.3, 1.4, or 1.5 kb.

In some embodiments, the promoter increases expression of the target gene, relative to expression of the target gene when using a vimentin promoter without the enhancer portion.

In some embodiments, wherein the core promoter portion is derived from a core region of minimal vimentin promoter (minVim). In some embodiments, the core promoter portion comprises the nucleic acid sequence of SEQ ID NO: 2 or its complement.

In some embodiments, the promoter increases expression of the target gene in astrocytes and decreases expression of the target gene in cells that do not express vimentin (e.g., MCF-7 cells).

In some embodiments, the core promoter portion is derived from a core region of a promoter selected from the group consisting of: minimal CMV promoter (minCMV), minimal simian virus 40 promoter (minSV40), minimal thymidine kinase promoter (miniTK), and YB TATA.

In some embodiments, the linker comprises the nucleic acid sequence of SEQ ID NO: 3.

In some embodiments, the promoter comprises the nucleic acid sequence of SEQ ID NO: 4.

The presently-disclosed subject matter includes a vector, which includes a promoter as described herein. In some embodiments, the vector is an adenovirus associated virus (AAV).

The presently-disclosed subject matter includes a method of modulating expression of a target gene in an astrocyte, which involves administering a vector for enhancing or suppressing expression of the target gene, which contains a promoter as described herein. In some embodiments, the vector is an adenovirus associated virus (AAV).

In some embodiments of the method, the target gene is involved in inflammatory or protective effects of reactive gliosis. In some embodiments of the method, the astrocyte is in a subject.

In some embodiments of the method, the subject has or has a risk of Alzheimer's disease or Parkinson's disease.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK© and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

### Example 1

A novel mutant of the AAV6 capsid was generated (rAAV.ShH10) that transduces Müller cells and, to a lesser extent, astrocytes, after an intravitreal injection (Klimczak et al, 2009). ShH10 mutations and an additional mutation were introduced into rAAV6 to yield rAAV6quad.

In order to provide astrocyte specific expression, a vimentin promoter obtained via Addgene (plasmid #29114). From this, a minimal vimentin promoter (mVim) was generated.

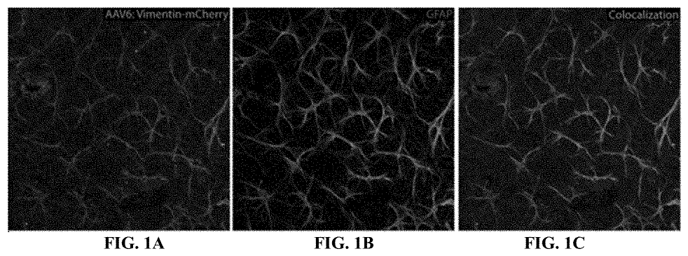

To test the cellular specificity of the vector in vivo mCherry was cloned downstream of mVim in the pAAV2 backbone, it was packaged into the rAAV6quad capsid, and it was injected intravitreally. Four weeks later, the retina was collected and flat-mounted, immunolabeled for GFAP and another astrocyte marker, and confocal microscopy was performed. Specific expression of mCherry was detected in the astrocytes, but not Müller glia (FIG. 1A-1C, results obtained with 2.7 kb promoter).

The results presented in FIGS. 1A-1C illustrate that the rAAV6quad.mVim specifically transduces astrocytes. These figures include Confocal micrographs of a retinal flat-mount after IVt injection of rAAVquadmut.mVim.mCherry (red in a color stain, but illustrated in grayscale herein) showing complete overlap with GFAP positive astrocytes (green in a color stain, but illustrated in grayscale herein) and no evidence of Müller cell endfeet. FIG. 1A is primarily stained red (red in a color stain, but illustrated in grayscale herein), while FIGS. 1B and 1C appear primarily green (green in a color stain, but illustrated in grayscale herein).

### Example 2

The design of an exemplary vimentin promoter upstream enhancer includes as follows. Plasmid VZ-60 includes 42 bp (−928 to −886) plus 24 bp linker plus 363 bp hVim “core” promoter. A 42 bp enhancer region is provided. A positive-acting region is provided for primary astrocytes, which are cells that express endogenous vimentin. A negative-acting region is provided for MCF-7 cells, which are cells that do not express detectable levels of vimentin

The sequence of the exemplary hVimentin Mini Promoter in Plasmid VZ-60 is as follows, provided with annotation.

(SEQ ID NO: 4)
AATCTGGTCTAACGGTTTCCCCTAAACCGCTAGGAGCCCTCAgctagca
tccggagagctcgaattCGGAAAGCCCCCAAAAGTCCCAGCCCAGCGCT
GAAGTAACGGGACCATGCCCAGTCCCAGGCCCCGGAGCAGGAAGGCTCG
AGGGCGCCCCCACCCCACCCGCCCACCCTCCCCGCTTCTCGCTAGGTCC
CTATTGGCTGGCGCGCTCCGCGGCTGGGATGGCAGTGGGAGGGGACCCT
CTTTCCTAACGGGGTTATAAAAACAGCGCCCTCGGCGGGGTCCAGTCCT
CTGCCACTCTCGCTCCGAGGTCCCCGCGCCAGAGACGCAGCCGCGCTCC
CACCACCCACACCCACCGCGCCCTCGTTCGCCTCTTCTCCGGGAGCCAG
TCCGCGCCACCGCCGCCGCCCAGGCCATCGCCACCGG
Underlined = 42 bp human vimentin “enhancer”
lower case bold = 24 bp linker (not central to
activity, but including restriction sites)
Remaining = 363 bp human vimentin “core” promoter

### Example 3

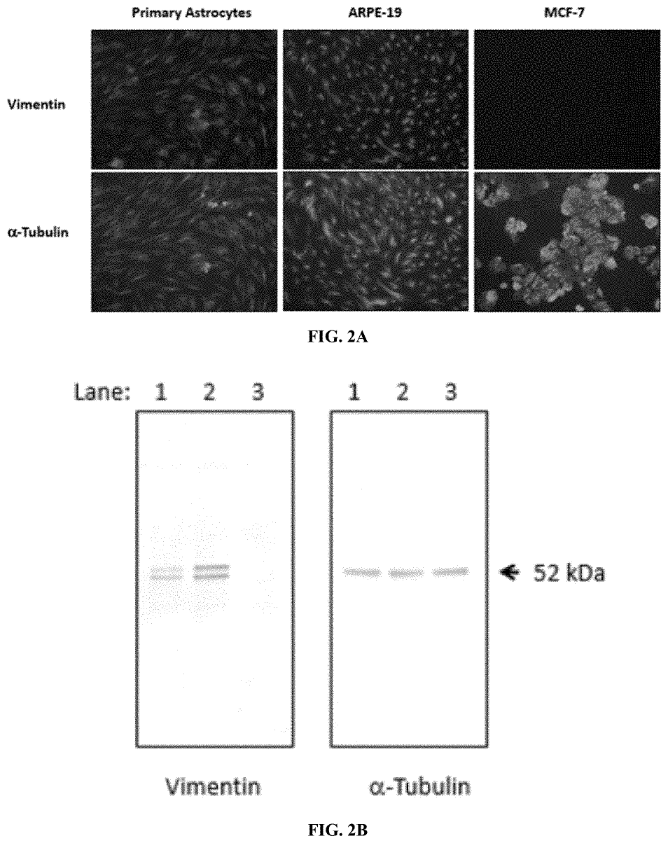

An analysis of endogenous vimentin expression was conducted. FIG. 2A includes a series of confocal micrographs, providing an analysis of endogenous vimentin expression. The first column of images are for mouse retinal primary astrocytes. The second column of images are for human retinal pigmented epithelia (ARPE-19). The third column of images are for human breast adenocarcinoma (MCF-7). In the first row, vimentin is visualized (red in a color stain, but illustrated in grayscale herein). In the second row, α-tubulin is visualized (green in a color stain, but illustrated in grayscale herein).

FIG. 2B includes Western blots, where 20 mg of cell extract was placed in each lane. Lanes indicated with 1 are for mouse retinal primary astrocytes. Lanes indicated with 2 are for human retinal pigmented epithelia (ARPE-19). Lanes indicated with 3 are for human breast adenocarcinoma (MCF-7). The left panel is vimentin and the right panel is α-tubulin. Vimentin expression levels were highest from ARPE-19 cells and below the level of detection from MCF-7 cells.

### Example 4

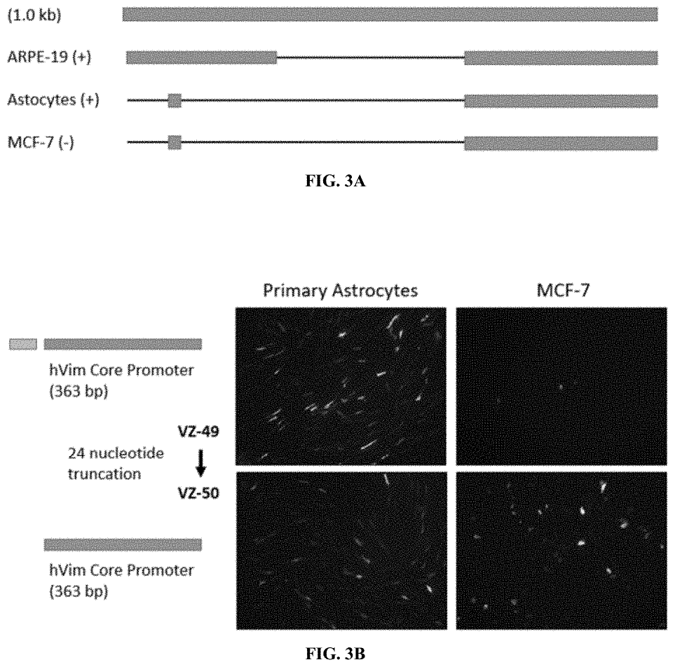

Regions that regulate expression from the human vimentin promoter (1.0 kb) in different cell types were studied. FIG. 3A includes depictions of promoter constructs. Cells were transiently transfected with different promoter constructs (grey regions) to study regions that regulate expression from the human vimentin promoter in different cell types. The promoter constructs for ARPE-19, astrocytes, and MCF-7 cells, relative to the human vimentin promoter (1.0 kb), are depicted.

The high expression level of endogenous vimentin from ARPE-19 cells (FIG. 2) is likely due to more positively-acting regions of the vimentin promoter (FIG. 3A, larger grey bar). With reference to FIG. 3B, the region that enhances expression (+) in primary mouse astrocytes decreases expression (−) in MCF-7 cells.

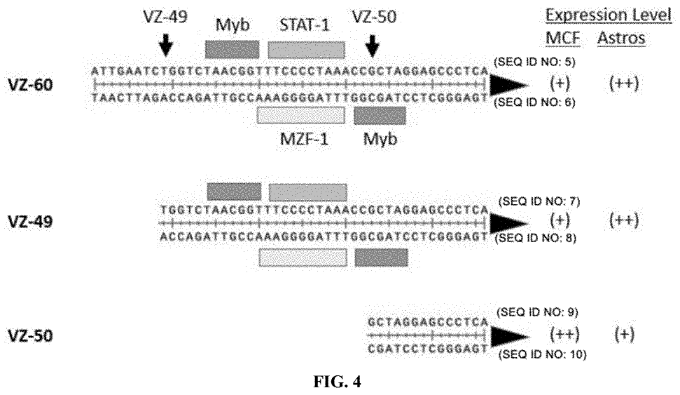

Various vimentin promoter truncations and there effect on expression and inhibition were studied. With reference to FIG. 4, vimentin promoter truncations with core promoter were studied, including VZ-60 (SEQ ID NOS: 5 and 6), VZ-49 (SEQ ID NOS: 7 and 8), and VZ-50 (SEQ ID NOS: 9 and 10). Expression levels in MCF-7 cells and astrocytes were obtained.

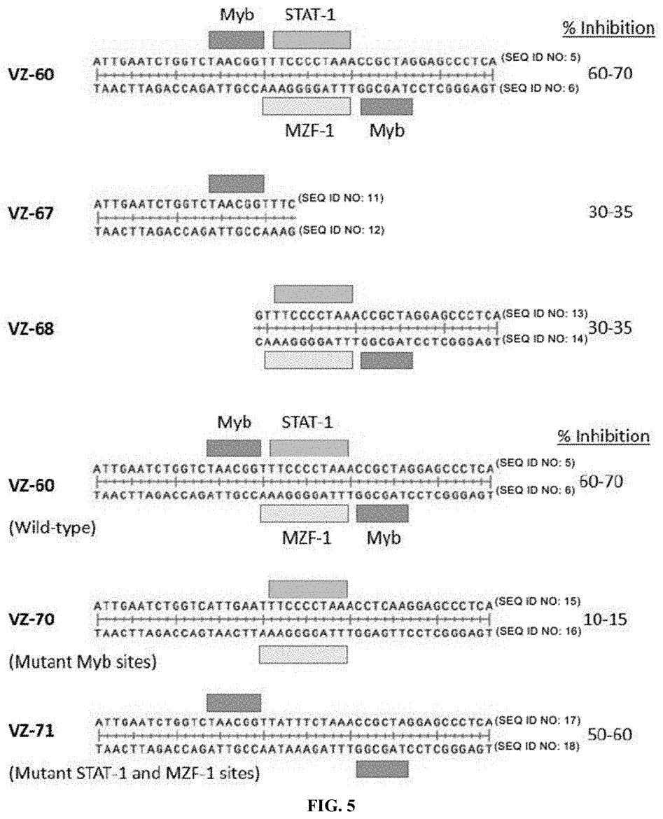

With reference to FIG. 5, analysis of vimentin promoters was conducted in MCF-7 cells, including VZ-60 (SEQ ID NOS: 5 and 6), VZ-67 (SEQ ID NOS: 11 and 12), and VZ-68 (SEQ ID NOS: 13 and 14, and also including VZ-60 (SEQ ID NOS: 5 and 6), VZ-70 (SEQ ID NOS: 15 and 16), and VZ-71 (SEQ ID NOS: 17 and 18). Percent (%) inhibition was determined.

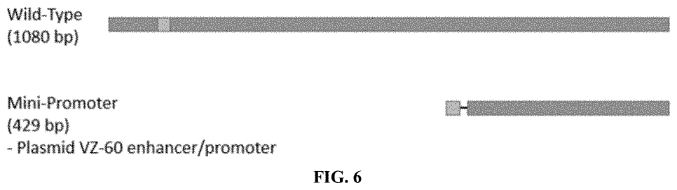

FIG. 6 includes a depiction of a wildtype human vimentin promoter (1080 bps), as compared to an example of a mini human vimentin promoter as disclosed herein. It is contemplated that, in some embodiments, the “core” vimentin promoter could be exchanged for another mini-promoter, such as, for example, minCMV, minSV40, miniTK, or YB_TATA.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Klimczak, R., Koerber, J., Dalkara, D., Flannery, J., and Schaffer, D., A novel adeno-associated viral variant for efficient and selective intravitreal transduction of rat Muller cells. PLoS One, 2009. 4: e7467.
2. O'Carroll Simon J., Cook William H., Young Deborah. AAV Targeting of Glial Cell Types in the Central and Peripheral Nervous System and Relevance to Human Gene Therapy. Frontiers in Molecular Neuroscience 2021; 13: 256. (doi:10.3389/fnmol.2020.618020).

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A promoter for modulating expression of a target gene from a vector, comprising:
an enhancer portion comprising the nucleotide sequence of SEQ ID NO: 1 or its complement;
a core promoter portion, containing an RNA polymerase binding site, a TATA box, and a transcription start site; and
a linker disposed between the enhancer portion and the core promoter portion, containing a restriction site.

2. The promoter of claim 1, wherein the promoter having the enhancer portion increases expression of the target gene in astrocytes or Muller cells, while decreasing expression of the target gene in other cells.

3. The promoter of claim 1, wherein the promoter increases expression of the target gene, relative to expression of the target gene when using a vimentin promoter without the enhancer portion.

4. The promoter of claim 1, wherein the core promoter portion is derived from a core region of minimal vimentin promoter (minVim).

5. The promoter of claim 4, wherein the core promoter portion comprises the nucleic acid sequence of SEQ ID NO: 2 or its complement.

6. The promoter of claim 4, wherein the promoter increases expression of the target gene in cells that express vimentin and decreases expression of the target gene in cells that do not express vimentin.

7. The promoter of claim 1, wherein the linker comprises the nucleic acid sequence of SEQ ID NO: 3.

8. The promoter of claim 1, comprising the nucleic acid sequence of SEQ ID NO: 4.

9. A vector comprising the promoter of claim 1.

10. The vector of claim 9, wherein the vector is an adenovirus associated virus (AAV).

11. A method of modulating expression of a target gene in a cell, comprising administering a vector for enhancing or suppressing expression of the target gene, containing the promoter according to claim 1.

12. The method of claim 11 wherein the vector is an AAV.

13. The method of claim 11, wherein the target gene is involved in inflammatory or protective effects of reactive gliosis.

14. The method of claim 1, wherein the cell is in a subject.

15. The method of claim 14, wherein the subject has or has a risk of Alzheimer's disease or Parkinson's disease.


SEQUENCE LISTING (18 sequences)

SEQ ID NO: 1 - DNA, 42 bp, synthetic construct
  source 1..42: mol_type = other DNA
aatctggtctaacggtttcccctaaaccgctaggagccctca

SEQ ID NO: 2 - DNA, 363 bp, synthetic construct
  source 1..363: mol_type = other DNA
cggaaagcccccaaaagtcccagcccagcgctgaagtaacgggaccatgcccagtcccaggccccggagcaggaaggctcgagggcgcccccaccccacccgcccaccctccccgcttctcgctaggtccctattggctggcgcgctccgcggctgggatggcagtgggaggggaccctctttcctaacggggttataaaaacagcgccctcggcggggtccagtcctctgccactctcgctccgaggtccccgcgccagagacgcagccgcgctcccaccacccacacccaccgcgccctcgttcgcctcttctccgggagccagtccgcgccaccgccgccgcccaggccatcgccaccgg

SEQ ID NO: 3 - DNA, 24 bp, synthetic construct
  source 1..24: mol_type = other DNA
gctagcatccggagagctcgaatt

SEQ ID NO: 4 - DNA, 429 bp, synthetic construct
  source 1..429: mol_type = other DNA
aatctggtctaacggtttcccctaaaccgctaggagccctcagctagcatccggagagctcgaattcggaaagcccccaaaagtcccagcccagcgctgaagtaacgggaccatgcccagtcccaggccccggagcaggaaggctcgagggcgcccccaccccacccgcccaccctccccgcttctcgctaggtccctattggctggcgcgctccgcggctgggatggcagtgggaggggaccctctttcctaacggggttataaaaacagcgccctcggcggggtccagtcctctgccactctcgctccgaggtccccgcgccagagacgcagccgcgctcccaccacccacacccaccgcgccctcgttcgcctcttctccgggagccagtccgcgccaccgccgccgcccaggccatcgccaccgg

SEQ ID NO: 5 - DNA, 46 bp, synthetic construct
  source 1..46: mol_type = other DNA
aatgaatctggtctaacggtttcccctaaaccgctaggagccctca

SEQ ID NO: 6 - DNA, 46 bp, synthetic construct
  source 1..46: mol_type = other DNA
taacttagaccagattgccaaaggggatttggcgatcctcgggagt

SEQ ID NO: 7 - DNA, 38 bp, synthetic construct
  source 1..38: mol_type = other DNA
tggtctaacggtttcccctaaaccgctaggagccctca

SEQ ID NO: 8 - DNA, 38 bp, synthetic construct
  source 1..38: mol_type = other DNA
accagattgccaaaggggatttggcgatcctcgggagt

SEQ ID NO: 9 - DNA, 14 bp, synthetic construct
  source 1..14: mol_type = other DNA
gctaggagccctca

SEQ ID NO: 10 - DNA, 14 bp, synthetic construct
  source 1..14: mol_type = other DNA
cgatcctcgggagt

SEQ ID NO: 11 - DNA, 23 bp, synthetic construct
  source 1..23: mol_type = other DNA
attgaatctggtctaacggtttc

SEQ ID NO: 12 - DNA, 23 bp, synthetic construct
  source 1..23: mol_type = other DNA
taacttagaccagattgccaaag

SEQ ID NO: 13 - DNA, 28 bp, synthetic construct
  source 1..28: mol_type = other DNA
gtttcccctaaaccgctaggagccctca

SEQ ID NO: 14 - DNA, 28 bp, synthetic construct
  source 1..28: mol_type = other DNA
caaaggggatttggcgatcctcgggagt

SEQ ID NO: 15 - DNA, 46 bp, synthetic construct
  source 1..46: mol_type = other DNA
attgaatctggtcattgaatttcccctaaacctcaaggagccctca

SEQ ID NO: 16 - DNA, 46 bp, synthetic construct
  source 1..46: mol_type = other DNA
taacttagaccagtaacttaaaggggatttggagttcctcgggagt

SEQ ID NO: 17 - DNA, 46 bp, synthetic construct
  source 1..46: mol_type = other DNA
attgaatctggtctaacggttatttctaaaccgctaggagccctca

SEQ ID NO: 18 - DNA, 46 bp, synthetic construct
  source 1..46: mol_type = other DNA
taacttagaccagattgccaataaagatttggcgatcctcgggagt